(19) United States
(12) Patent Application Publication
(10) Pub. No.: US 2014/0080773 A1
(43) Pub. Date: Mar. 20, 2014

(54) Composition for Delivering an Anti-Ageing Effect on the Skin and a Method for Improving Skin Characteristics

(71) Applicant: Conopco, Inc. d/b/a UNILEVER, Englewood Cliffs, NJ (US)

(72) Inventors: Gail Jenkins, Huntingdon (GB); John Casey, Yeldon (GB); Julia Sarah Rogers, Sharnbrook (GB)

(73) Assignee: Conopco, Inc. d/b/a UNILEVER, Englewood Cliffs, NJ (US)

(21) Appl. No.: 13/969,736

(22) Filed: Aug. 19, 2013

Related U.S. Application Data
(60) Provisional application No. 61/702,928, filed on Sep. 19, 2012.

(51) Int. Cl. A61K 8/67; A23L 1/29; A61K 8/36; A61Q 19/08; A61K 8/60; A61K 8/31

(52) U.S. Cl. CPC A61K 8/678; A61K 8/602; A61K 8/676; A61K 8/31; A61K 8/361; A61Q 19/08; A23L 1/296
USPC 514/27

(57) ABSTRACT

A composition suitable to improve skin characteristics is described along with a regimen to improve skin characteristics. The composition is not limited to format, and can be in the form of a capsule, tablet or food product. When consumed within a defined regimen, the composition with active mixture results in skin that is smoother and with a reduction in wrinkles.

### FIELD OF THE INVENTION

The present invention is directed to a composition suitable for oral consumption and a method for improving skin characteristics with such composition. More particularly, the invention is directed to a composition that preferably is within a capsule or in tablet form (for easy consumption) whereby such composition is suitable for consumption via a regimen to improve skin characteristics.

### BACKGROUND OF THE INVENTION

Improving the appearance and feel of human skin has received a great deal of research effort. This research effort is a direct response to both men and women wanting to look and feel younger. The vast majority of commercially available products address skin appearance and aging typically by acting on the exterior of the skin. The most common forms being topical skin creams or lotions.

While topical creams and lotions are popular, such topical products have limitations and deal primarily with dead surface (external) layers of the skin.

It is known that certain ingredients can provide improvements in skin appearance and texture subsequent to being ingested. Such ingredients thus act from the interior of the skin, and therefore, can provide additional opportunities for improving the skin by accessing the living interior. Furthermore, such an effect may be perceived by the general public as being more potent or medical in nature than a topical application.

Examples of ingredients that benefit skin include dietary fish oils. The same are known to convey significant protection against UVR-induced erythema upon ingestion. Carotenoids, such as lycopene and β-carotene, have also been shown to give significant protection against UVR-induced erythema when consumed orally. Likewise, vitamins E & C when taken orally have also been shown to provide protection against UVR-induced erythema.

While known creams, lotions and ingestible ingredients have been made available to improve skin characteristics, products that are easy to ingest and provide enhanced benefits are desired by consumers, and especially, those that may be taken via an easy to follow regimen.

This invention, therefore, is directed to a composition suitable for oral consumption and to a method for improving skin characteristics with such composition. More particularly, this invention is directed to a composition that preferably is within a capsule or in tablet form whereby such composition is suitable for consumption via a daily regimen that unexpectedly results in an excellent improvement of skin characteristics.

### ADDITIONAL INFORMATION

Efforts have been disclosed for making consumable products suitable to provide benefits to consumers. In U.S. Pat. No. 6,589,535, disclosed is a nutritional supplement which contains an oil rich in ω-3 and ω-6 fatty acids and a carotenoid in combination to combat the harmful effects of xenobiotics on the skin, in particular on the skin's immune system.

Other efforts have been disclosed that describe consumable compositions for providing benefits to consumers. In U.S. Published Patent Application No. US2003/0082275, disclosed is a drinkable ω-3 preparation which is storage stable.

Even other efforts have been disclosed for making consumable products that provide benefits to consumers. In World Applications 20021074308 and 20061056293 as well as in U.S. Pat. Nos. 6,060,070 and 5,976,606, and EP-A-1340427 and DE-U-20304752, consumable compositions geared to provide consumer benefits are described.

Finally, in U.S. Pat. Nos. 7,723,537, 7,659,234 and 7,659,234, topical skin benefit compositions are described.

None of the additional information mentioned above describes a composition that is suitable for consumption via a daily regimen to provide excellent skin benefits as described in this invention.

### SUMMARY OF THE INVENTION

In a first aspect, the present invention is directed to a composition comprising:

a) a carotenoid;

b) a PPAR ligand;

c) an oestrogen receptor binding agent;

d) an agent involved in post translational modification of collagen; and

e) an antioxidant

wherein the agent involved in post translational modification of collagen and antioxidant are present at a daily dosage amount (x) that is from about 0.02 to about 26 times a daily dosage amount (y) that is the daily dosage amount of carotenoid, PPAR ligand and oestrogen receptor binding agent in the composition further wherein the antioxidant daily dosage amount carotenoid daily dosage amount within the composition is from about 0.007 to about 620, the composition being one capable of delivering from about 45 to about 2000 mg PPAR ligand, from about 0.5 to about 120 mg carotenoid, from about 8 to about 125 mg oestrogen receptor binding agent, from about 45 to about 1100 mg of an agent involved in post translational modification of collagen and from about 0.85 to about 310 mg antioxidant in about a daily dosage.

In an often preferred embodiments, (x) is from about 0.04 to about 5.3 times the daily dosage amount (y), and the antioxidant daily dosage amount/carotenoid daily dosage amount within the composition is from about 0.33 to about 400.

In a second aspect, the present invention is directed to a method for providing an anti-aging effect to skin by consuming the composition described in the first aspect of this invention.

All other aspects of the present invention will more readily become apparent upon considering the detailed description and examples which follow.

Skin, as used herein, is meant to include skin on the face, neck, chest, back, arms (including underarms), hands, legs, buttocks and scalp. Excellent improvement of skin characteristics means that often visible skin benefits in terms of smoother skin, and skin line and wrinkle reduction after consuming the composition of this invention, via a defined regimen. Such improvement of skin characteristics includes an anti-ageing effect in the skin of a human or non-human mammal (preferably a human), which comprises providing the human or non-human mammal with an amount of a composition of the invention which is effective to achieve the aforementioned improvement in skin characteristics. The invention described herein further provides a method of increasing collagen synthesis in the skin of the mammal (preferably a human) which comprises providing the human or non-human mammal with an amount of composition of the invention which is effective to deliver a dosage of active suitable to achieve such result. Typically, it is expected that the effects of the use of the composition of this invention will be measured to have a significant improvement after establishing a defined regimen which is about a daily use or dosage (i.e., consumption) for about ten (10) weeks to about sixteen (16) weeks, and preferably, from about twelve (12) weeks to about fifteen (15) weeks. About a daily use or dosage is meant to mean a certain or defined amount of composition consumed one (1) time (as to the total daily dosage) every one or two days but preferably everyday. If, for example, a regimen mils for 10 milligrams of active A as the daily dosage, in this invention the same includes within the meaning of daily dosage 10 milligrams of active A taken at one time daily or a total of 10 milligrams taken over multiple times (e.g., such as consuming 2 milligrams of active 5 times over the course of such day). Composition, as used herein, is meant to mean an end use composition such as capsule, tablet, drink or snack bar. By illustration, composition as used herein as it relates to, for example, one capable of delivering 100 mg carotenoid in about a daily dosage is meant to include, for example, one tablet with 100 mg of caretonoid, or one tablet with 50 mg of caretonoid plus a snack bar with 25 mg of caretonoid plus a drink with 25 mg of carotenoid and so on. The about daily dosage can be achieved, therefore, with one or multiple composition consumptions every one or two days within the regimen.

Composition, as used herein, means the end use product comprising the mixture consumed by the consumer that comprises active. The composition of this invention is often from about 10 to about 100%, and preferably, from about 15 to about 95%, and most preferably, from about 20 to about 85% by weight active, based on total weight of the composition. When the composition of this invention is in a gel or tablet format, for example, excipient often makes up from about 0.75 to about 45%, and preferably, from about 5.0 to about 40%, and most preferably, from about 15 to about 38% by weight of the total weight of the composition

Isoflavone aglycone equivalents are calculated by multiplying the aglycone, glucoside, acetyl glucoside, and malonyl glucoside concentrations for each isoflavone by their respective conversion factors (based on molecular weight) and adding together the results. Aglycones are equivalent to 0.625 times total glucoside Isoflavones.

Comprising, as used herein, is meant to include consisting essentially of and consisting of. For the avoidance of doubt, therefore, comprising caretenoid, PPAR ligand, oestrogen receptor binding agent, agent involved in post translational modification of collagen; and antioxidant includes a composition consisting essentially of the same as well as a composition consisting of the same. Active, as used herein, means an ingredient that provides a benefit to skin either alone or in combination with another ingredient. Ingredients (a) through (e) as set forth in the first aspect of the invention are illustrative actives suitable to mix (forming a mixture of actives) for use in the composition of this invention. All ranges included herein are meant to include all ranges subsumed herein unless stated otherwise to the contrary.

### DETAILED DESCRIPTION OF THE PREFERRED EMBODIMENTS

### The PPAR Ligand

Peroxisome proliferator-activated receptors (abbreviated herein as PPAR) are transcription factors that control lipid metabolism. PPAR ligands are known and are described, for example, in WO 02/102337, the contents of which are incorporated herein by reference. The only limitation with respect to the type of PPAR ligands that may be used in this invention is that the same are suitable for consumption by humans.

Preferably, the PPAR ligand comprises an omega-3 fatty acid (i.e., an unsaturated carboxylic acid having from 12 to 26 carbon atoms). Preferred omega-3 fatty acids are those selected from DHA, EPA and mixtures thereof.

Typically, and consistent with this invention, from about 45 to about 2,000 mg, and preferably, from about 100 to about 1,400 mg, and most preferably, from about 400 to about 800 mg of omega-3 fatty acid is the dosage consumed about daily as provided from the composition with active mixture of this invention (and including all ranges subsumed therein).

The omega-3 fatty acid is preferably present in the form of a fish oil or is from a microbial source. The omega-3 fatty acid may be in the form of a free acid, a C1 to C6 alkyl ester, a glyceride (including mono-, di- and tri-glycerides) or mixtures thereof. Preferably, the omega-3 fatty acid is in the form of a glyceride (e.g., a triglyceride). Reference herein to the omega-3 fatty acid means the free acid or alkyl esters or glycerides or mixtures thereof.

Preferred omega-3 fatty acids are, again, DHA and EPA.

DHA is an ω-3, polyunsaturated, 22-carbon fatty acid. It is also present in abundance in certain fish (such as tuna and bluefish) and marine animal oils.

DHA may optionally be present together with EPA.

Eicosapentaenoic acid (EPA) is one of several ω-3 fatty acids used by the body. Increased intake of EPA has been shown to be beneficial in coronary heart disease, high blood pressure, and inflammatory disorders such as rheumatoid arthritis.

Eicosapentaenoic acid (EPA) and docosahexaenoic acid (DHA) come from cold water fish such as wild salmon, mackerel, sardines, herring and other northern marine animals. Fish can make EPA and DHA from the ω3 essential fatty acid, alpha-linolenic acid (LNA), but get much of their EPA and DHA from brown and red algae which manufacture EPA and DHA from carbohydrates—sugar, starch, cellulose, etc.

More recently, brown and red algae have begun to be grown commercially for EPA and DHA. These make 10 to 14% of long-chain ω3s (on dry weight basis) and can be used as food sources of EPA and DHA-containing triglycerides.

When both DHA and EPA are present in the composition of the invention, the weight ratio of DHA to EPA is typically from about 1:10 to 10:1, and preferably, from about 5:1 to 1:5, and most preferably, from about 3:1 to 1:3, with a 1:1 to 1:2 or a 3:2 to 2:3, or a 4:3 or 3:4 weight ratio often being especially preferred.

### The Oestrogen Receptor Binding Agent

The composition of the present invention and comprising active mixture further comprises an oestrogen receptor binding agent. The oestrogen receptor binding agent is preferably a natural product or a derivative or extract thereof.

Preferably, the oestrogen receptor binding agent comprises one or more soy isoflavones. The preferred soy isoflavone is genistein or daidzein or a mixture thereof. Most preferably, the soy isoflavone is genistein.

The composition of the present invention often comprises oestrogen receptor binding agent in an amount such that an about daily dosage delivers from about 8 to about 125 mg, and preferably, from about 10 to about 70 mg, and most preferably, from about 15 to about 45 mg, including all ranges subsumed therein.

The oestrogen receptor binding agent may be in glycosylated or non-glycosylated form, or a mixture of these two forms. Reference to the same herein means the glycosylated or non-glycosylated forms, or mixtures of the two forms, unless specifically stated otherwise. Such agent is preferably present in the composition of the invention as a component of a natural product or an extract or concentrate thereof. Preferably, the natural product is soy or red clover, more preferably soy. In an especially preferred embodiment, the oestrogen receptor binding agent is in the glycine equivalent form such that from about 3 to about 85%, and preferably, from about 4 to about 75%, and most preferably from about 5 to about 65% by weight of all oestrogen receptor biding agent is in the aglycone equivalent form.

To the extent genistein is used in this invention, the same, when it is from soy, is preferably purified at least to some extent by removal of soy protein. Therefore, compositions of the invention preferably contain less than 1% by weight of soy protein, and preferably, less than 0.5% by weight of soy protein, and most preferably, less than 0.1% by weight of soy protein, such as less than 0.01% or less than 0.001% or less than 0.0001% by weight. The composition and active mixture of the invention may, therefore, be free of soy protein or substantially free of soy protein.

The Agent that is Involved in the Post-Translational Modification of Collagen

The composition of the invention with active mixture can comprise a component that is an agent (e.g., compound) involved in the post-translational modification of collagen. Preferably, the agent is a cofactor in the hydroxylation of proline residues.

Preferably, the agent that is involved in the post-translational modification of collagen is vitamin C.

Typically, vitamin C is present in composition of the present invention in an amount such that an about daily dosage of composition delivers from about 45 to about 1100 mg, and preferably, from about 60 to about 400 mg, and most preferably, from about 150 to about 250 mg.

### The Carotenoid

The composition of the invention with active mixture comprises one or more carotenoids.

Typically, caretonoid is present in the composition of this invention in an amount such that an about daily dosage of composition delivers from about 0.5 to about 120 mg, and preferably from about 1.0 to about 60 mg, and most preferably, from about 1.0 to about 10 mg. Preferably, the carotenoid is selected from P.-carotene, lycopene or a mixture thereof.

### The Antioxidant

At least one antioxidant is typically present in the composition with mixture of actives of this invention. Illustrative examples of the antioxidants suitable for use include either singularly or in combination: tertiary butylhydroquinone (TBHQ), ascorbyl esters (e.g. ascorbyl palmitate), ascorbic acid, tocopherols, rosemary extract, fruit concentrates or extracts, black or green tea extract, oropyl gallate, essential oils or oleoresins, butylated hydroxyanisole (BHA), butylated hydroxytoluene (BHT), citric acid or esters thereof, ubiquinone, tocotrienols, chelators (e.g. EDTA), carriers, polyphenols, phenolic compounds, flavonoids, oxygen scavengers (e.g. glutathione, ascorbic acid, cysteine) mixtures thereof or the like.

An especially preferred antioxidant suitable for use in this invention is vitamin E.

The amount of Antioxidant used in this invention is such that about a daily dosage of composition delivers from about 0.85 to about 310 mg, and preferably, from about 1.0 to about 175 mg, and most preferably, from about 10 to about 45 mg antioxidant.

### Optional Components

The composition of the present invention may comprise one or more optional components. Preferred optional components include those selected from flavouring agents, (e.g., sweeteners), preservatives, colorants or combinations thereof.

Suitable flavouring agents may be natural or synthetic. Flavouring may be required to make the product more palatable for consumption. Artificial and/or natural flavouring agents may be used with natural flavouring agents being especially preferred. Certain optional ingredients also can include ingredients typically applied to skin topically. It is also within the scope of the invention to include within the composition art recognized thickeners (e.g., gums, waxes), stabilizers, emulsifiers, amino acids like taurine, extracts from plants (e.g., sage, chamomile and yarrow extracts, green tea extract, caffeine, grape seed extract, pine bark extract, as well as extracts classified as an extract of Sanguisorbo officinalis); mixtures thereof or the like.

Preservatives suitable for use in the compositions of this invention include those generally classified as food grade. Illustrative examples include sodium benzoate, tetrasodium pyrophosphate, potassium, sorbate, mixtures thereof or the like. Colorants suitable for use include those generally classified as FD&C lakes. Typically, the compositions of the present invention comprise from about 0.0 to about 12%, and preferably, from about 0.001 to about 8.0% by weight optional additive, based on total weight of the composition and including all ranges subsumed therein.

Ingredients suitable for use herein that are often found topically applied to skin include niacinamide, vitamin A and its derivatives, conjugated linoleic acid, petroselinic acid, mixtures thereof or the like. When employed, the daily dosage of the same should not exceed about 2000 mg per day collectively, and preferably, an about daily dosage of from about 0.0001 to about 1000 mg, and most preferably, from about 0.005 to about 750 mg about daily is consumed, all based on a ten (10) to sixteen (16) week regimen period and with the proviso that when niacinamide is used the daily dosage of the same preferably does not exceed 25 mg and often is from about 0.00001 to about 20 mg per day for the same period.

The extracts and amino acids suitable for use herein, when used, typically and individually are included to deliver an about daily dosage of about 10 to about 300 mg within the regimen of this invention and including all ranges subsumed therein.

Composition of the present invention is typically consumed from one (1) to ten (10) times daily, and preferably, one (1) to seven (7) times daily, and most preferably, one (1) to five (5) times daily in order to ensure that the consumer consumes the appropriate daily dosage of active ingredients within the regimen period in order to achieve the unexpected and superior results described.

The composition of the present invention is preferably free from added zinc and/or selenium. The compositions of the invention may contain trace amounts of zinc and/or selenium from the commercially available components of the composition but preferably do not contain added zinc or selenium in the form of their metals or salts. Thus, the compositions of the invention may, for example, contain less than 0.5 mg zinc and/or less than 0.01 mg selenium or less than 0.0005% zinc and less than 0.00001% selenium, and more preferably, less than 0.0001% zinc and less than 0.000005% selenium.

### Product Form

The composition of the present invention is edible and is suitable to be combined with a liquid like water. If a liquid is used, in a preferred embodiment, the liquid should make up from about 50 to about 99%, and most preferably, from about 55 to about 95%, and optionally, from about 70 to about 90% by weight of the composition (i.e., end use and consumed), including all ranges subsumed therein.

The composition of the present invention may also be carried in a food product or nutritional supplement. Compositions for oral consumption which may be used according to the invention include beverages (like fruit drinks), bars (like snack bars) and other liquid and solid forms such as capsules or tablets and even powders (which may contain crystalline material), as well as spreads, margarines, creams, sauces, dressings, mayonnaises, ice creams, fillings, confectionaries and cereals.

Preferably, the composition of the present invention is in the form of a substantially homogeneous capsules or tablets that comprises less than about 10%, and preferably, from about 0.0001 to about 7% by weight water, and most preferably, from about 0.001 to about 6% by weight water based on total weight of the capsule or tablet and including all ranges subsumed therein. When making such capsules or tablets, conventional techniques should be used and as previously stated, excipient typically makes up from about 0.75 to about 45%, and preferably, from about 5.0 to about 40%, and most preferably, from about 15 to about 38% by weight of the composition, including all ranges subsumed therein. Typically, when a capsule is desired, the same has a core (center) and clad (coating or shell). In tablets, excipients can include talc, anti-adherents like magnesium stearate, binders like starches and cellulose as well as coatings such as hydroxypropyl methylcellulose. In the case of capsules (including soft capsules), shell ingredients can include gelling substances like collagen, gelatine and humectants like glycerine and sorbitol. When capsule is desired, the core typically makes up from about 65 to about 80% by weight of the capsule (or composition) with shell typically making up from about 20 to about 35% by weight of the composition.

The composition of the present invention, if desired, can be prepared from an aqueous phase and for an oil phase. Emulsifier, like food grade phospholipids, may be used and typically from about 0.05 to about 4%, and preferably, from about 0.05 to about 2% by weight emulsifier is used based on total weight of the phases.

The two phases can then be blended together in conventional emulsifier equipment. The resulting emulsion may be added to excipient for capsule or tablet formation. The resulting end use product or composition, if in capsule or tablet form, should be of a size that is both easy to swallow yet large enough for human fingers to handle. If a food product like a small drink or snack bar is desired, size of the same will be determined based on taste and the number of servings desired in order to reach the recommended daily dosage with the end use composition.

Another option is to simply combine core ingredients with or without wax and heat, and coating the same with conventional shell or clad ingredients.

### Uses of the Invention

The composition of this invention, when following the defined regimen, will produce an anti-ageing effect on skin. By the term “anti-ageing”, it is meant, again, that the skin may appear less wrinkled (i.e., there is an anti-wrinkling effect on wrinkles and/or fine lines, including a reduction in wrinkle depth) and that the composition may impart one or more further benefits for the skin selected from: reduced dryness; increased firmness; increased elasticity; increased smoothness; clearer skin; fewer spots, pimples and blemishes (including acne); less sensitive skin; and generally healthier skin. The composition may be provided with instructions on how to complete the regimen described herein.

Compositions of the invention may exhibit the anti-ageing effect by increasing collagen synthesis in the skin and compositions of the invention may be used to increase collagen synthesis (as part of, or separately from, the anti-ageing effect); preferably collagen synthesis is increased by at least 10%, more preferably at least 20% such as at least 25% by weight (e.g., as determined based on the weight of collagen synthesised, preferably over the regimen period).

The following non-limiting examples further illustrate the invention. They are provided to facilitate an understanding of the invention and not to limit the scope of the claimed invention.

### Analysis

The effectiveness of the combination was based on the degree of change in fine lines and wrinkle depth as measured by PRIMOS and the change in collagen synthesis as measured by enzyme immunoassay or visual histochemical analysis.

### Determination of Increase in Collagen Synthesis

### Outline of Experimental Approach

A biochemical assay and protein extraction method was developed to determine changes in new collagen synthesis in the skin.
a. Skin biopsies were taken at baseline (T1) and end (T15) of the intervention period.
b. At each time point, two 3 mm punch biopsies (4 mm depth) were taken, placed in a cryotube container and immediately snap frozen in liquid nitrogen.
c. These biopsies were then stored at −80° C.

### Materials and Methods

### Preparation of Cell Lysate

All punch biopsies were placed in a dounce homogenizer with 1 ml cell lysis buffer and ground up completely (so as no significant lumps of skin or extracellular matrix remained). The lysis buffer contained 1% NP-40, 0.1% sodium deoxycholate, 0.1% SDS, 6 mM sodium chloride and 0.05M Tris at pH T6. Protease inhibitor cocktail (1000×; Sigma P8340) was added prior to use at a level of 10 μl per ml of lysis buffer. Following complete homogenisation of the tissue, unwanted cell debris was removed by centrifugation for 20 minutes at 20,000 g at 4° C. The clarified cell lysate was frozen at 80° C. until needed,

### Total Protein Assay (Pierce)

The total protein concentration of each cell lysate was measured using the Pierce BCA protein assay kit. A set of eight standard solutions ranging from 0 to 1200 μg/ml protein was prepared from the supplied 2 mg/ml BSA stock solution. 10 μl of standard or cell lysate was added to duplicate wells of a flat-bottomed, 96-well microtitre plate. The reagent solution was prepared according to the kit instructions from 50 parts reagent A and 1 part reagent B. 200 μl of the final reagent was added to each well of the microtitre plate. The plate was mixed, covered and incubated at 37° C. for 30 minutes and absorbance read at 562 nm. A protein standard curve was constructed and used to determine the protein concentration of each cell lysate.

### Procollagen I C-Peptide EIA KIT (Takara Bio Inc.)

Collagen I is synthesised as a precursor molecule, Procollagen I. The amount of free propeptide therefore, reflects stoichiometrically, the amount of collagen I synthesised. The Procollagen Type I C-peptide Enzyme Immunoassay (EIA) kit allows for the quantitative determination of Procollagen Type I C-peptide (PIP).

Eight PIP standards were prepared in sample diluent at concentrations ranging from 0 to 640 ng/ml. 100 μl of antibody-Peroxidase conjugate solution and 20 μl of cell lysate (1 μg protein) or standard was added to duplicate wells. The plate was sealed and incubated at 37° C. for 3 hours before being washed four times with 400 μl of PBS. Each well then received 100 μl of substrate solution and the plate incubated at room temperature, on the benchtop, for 15 minutes. After this period, 100 μl stop solution was added to each well and absorbance measured at 450 nm with a plate reader.

A standard curve was plotted of mean absorbance versus PIP concentration and the line of best fit calculated by regression analysis. The unknown concentration of PIP in all the samples was estimated from this.

### Measurement of Skin Hydration

Various methods for determining the hydration state of the stratum comeum have been summarized by Fluhr et al., Skin Res Technol 1999; 5:161-170. Briefly, the Comeometer (Courage & Khazaka) measures skin hydration through detection of epidermal capacitance. The probe is made of two finger-type metal plates close to each other, with a measurement depth of approximately 30 mm. The instrument determines the humidity level of the most external cutaneous layers of the stratum comeum. The action principle of the Comeometer® is based on the modification of the electrical capacities of the detector which is designed in the form of a condenser. The surface of the measurement head, in contact with the skin, modifies its electrical rapacity according to the humidity level of the skin. An increase in the value measured by the comeometer is indicative of improved skin hydration.

### Measurement of Trans Epidermal Water Loss (TEWL)

An analysis of methods to measure TEWL has been performed by Wilson & Maibach, (1989) Transepidermal water loss, A review, In: Cutaneous Investigation in Health and Disease, Non-invasive Methods and Instrumentation (Leveque, J. L., ed.), pp. 113-130, Dekker, New York, NY. The cutaneous barrier acts as a regulator in skin water balance. When this is damaged, the water exchange regulation system becomes destabilised. This means that water migrates more easily to the outside environment, increasing Transepidermal Water Loss. The effectiveness of the cutaneous barrier decreases with age. However, if the condition of the cutaneous barrier improves, water loss decreases as the water exchange regulation mechanism recovers its balance. TransEpidermal Water Loss measurements can be performed with a Servomed “Evaporimeter” EP-3®. A probe made up of two captors is traversed by a flow of water vapor. The difference of the partial pressure is measured between the two captors. This value corresponds to the evaporation speed of a volatile substance (in this case, water). A reduction in TEWL is indicative of improved skin barrier properties

### Measurement of Skin Elasticity & Firmness

Measurements for skin elasticity and firmness are made with a cutometer and described in Escoffier at al, J Invest Dermatol, 93(3):353-7. The measurement is done with an instrument which, using the vacuum principle, sucks up a defined area of skin surface and records it optically. Analysis of the recorded measurement curves makes it possible to determine the elastic and plastic characteristics of the skin. Young skin shows a high degree of elasticity and loses shape only gradually while regaining its original state after the end of the suction procedure. Skin which is young, healthy, supple and adequately moist will have a higher elasticity than an aged dry, rough skin. The cutometer therefore gives a set of measurements which allows us to quantify elastic characteristics. The technique consists of skin aspiration by a measurement probe. The skin is sucked into the orifice of the probe by negative pressure created within the device. The depth to which the skin penetrates into the probe is measured by a non-contact optical measurement system. This system consists of a light source and light receptor, as well as two prisms facing each other, which project the light from transmitter to receptor. Light intensity varies with penetration depth of the skin. The resistance of the skin to be sucked up gives an indication of the firmness of the skin and the ability to return to its original position gives an indication of the elasticity of the skin. A curve is displayed at the end of each measurement which allows several calculations to be made corresponding to skin mechanical properties.

### Histochemical Analysis of Collagen Synthesis

Punch biopsies, at baseline and following 14 weeks intervention, were obtained from the inner upper arm under 1% lignocaine local anaesthesia. Biopsied tissue was embedded in optimal cutting temperature compound (Tissue-Tex®; Miles Laboratories, Elkhart, Ind., USA), snap frozen in liquid nitrogen and stored at −70° C. prior to immunohistochemical analyses. The biopsies were sectioned, stained with H&E (hematoxylin and eosin stain) and evaluated by an independent histopathologist for changes in the quality and quantity of collagen.

### Analysis of Fine Lines, Wrinkles & Skin Smoothness

Skin roughness and wrinkling can be assessed using replicas and skin profilometry as described by Cook, J Soc Cosmet Chem, 1980; 31:339-359. A silicon rubber material such as Silflo is prepared and applied to the test area. Once set it is removed and analysed using optical profilometry. With this measurement method, a parallel stripe pattern is projected onto the skin surface and depicted on the COD chip of a camera. The 3D measurement effect is achieved by the fact that minute evaluation differences on the skin surface deflect the parallel projection stripes and that these deflections constitute a qualitative and quantitative measurement of the skin profile. The skin profiles are recorded by the CCD camera, digitized, and transferred to the measurement and evaluation computer for qualitative evaluation.

### EXAMPLES 1-4

The following Examples represent active mixtures consistent with this invention and that were tested clinically.

| | Example | Example | Example | Example |
|---|---|---|---|---|
| | 1 | 2 | 3 | 4 |
| Ingredients | Daily Dosage (mg) | | | |
| DHA and EPA* | 3000 | 2000 | 660 | 660 |
| Soy isoflavone glucoside)** | 98 | 98 | 69 | 40 |
| Vitamin C | 500 | 250 | 250 | 180 |
| Vitamin E | 500 | 250 | 250 | 30 |
| Lycopene (20% active) | 16 | 10 | 8 | 3 |
| Beta-carotene (30%) | 7 | 7 | — | — |

*ratio-Examples 1-3 at 4:3 and Example 4 at 3:2
**Isoflavone aglycone equivalents are calculated by multiplying the aglycone, glucoside, acetyl glycoside, and malonyl glucoside concentrations for each isoflavone by their respective conversion factors (based on molecular weight) and adding the results. Aglycones are equivalent to 0.625 times total glucoside isoflavones.
Examples 1 and 2: 61 mg aglycones;
Example 3: 43 mg aglycones;
Example 4: 25 mg aglycones.

The mixtures were prepared by adding the ingredients together and mixing to yield a homogeneous mixture. The mixture was added to capsule excipients to yield capsules, snack bars (1.5-3 ounces) or to a small fruit drink (4-6 ounces). Enough mixture was added so that the recommended about daily dosage was consumed either at one time or over the course of a few servings (e.g., with one bar and 8 capsules in a single day for the regimen period).

### EXAMPLE 2

End use compositions (capsules/drinks/bars) consistent with the invention were tested over 14 weeks via the method described above wherein the same yielded the results as presented below: All participants in the clinicals were instructed to consume the recommended daily dosages everyday for the fourteen (14) week trial period.

### EXAMPLE 5

Example 5 shows the unexpected and superior results obtained by the panellists/consumers that participated in the trials. All trials were for a fourteen (14) week period. The effectiveness of the composition was based on the change in wrinkle depth and degree of collagen synthesis.

Clinical (A): This clinical was conducted in France. One hundred and one (101) female consumers were given one small fruit drink and eight capsules per day to reach the recommended daily dosage consistent with this invention. About one half (½) of the consumers were given the capsules and drink containing active mixture consistent with the one described in Example 1, and one half (½) were given placebo.

After the fourteen week period was complete, it was unexpectedly found that the individuals that did not receive placebo showed significantly reduced wrinkle depth, a smoother skin surface and increased collagen synthesis.

Clinical (B): This clinical was conducted in Scotland. The procedure for Clinical A was essentially repeated for Clinical B except that one hundred and fifty-nine (159) women were tested, circa one third (⅓) were given a composition having the mixture of Example 1, one third (⅓) were given a composition having the mixture of Example 2 and one third (⅓) were given placebo (carried out in Scotland). Increases towards reduction in wrinkle depth and collagen synthesis were observed but the results were not significant against the placebo. It is believed that the results obtained in this clinical are a direct result of diet of the consumers tested as well as the season (Spring) for which wrinkles and facial lines tend to be less pronounced.

Clinical (C): Clinical C was conducted in a manner similar to the one described in Clinical A except that one hundred and thirty-six (136) female consumers participated. Circa one third (⅓) were given a composition having the mixture of Example 3, one third (⅓) were given a composition having the mixture of Example 4 and one third (⅓) were given placebo (carried out in Germany). Individuals given the composition consistent with the dosage requirements in this invention showed significantly reduced wrinkle depth and a smoother skin surface after fourteen (14) weeks. In this clinical one (1) fruit drink and two (2) capsules were taken daily but with the appropriate dosage adjustments to meet the daily amount suggested in this invention.

Clinical (D): Clinical D was conducted in a manner similar to the one described in Clinical A except that a single fruit drink of half the volume of the drink used in Clinical A was consumed by the consumers participating. The daily dosage was added, therefore, to the single beverage. Fifty (50%) of the consumers (about 170 panelists tested) were given composition with the mixtures of Example 4 and fifty (50%) were given placebo.

The results obtained after fourteen (14) weeks unexpectedly revealed a significant reduction in wrinkle depth and smoother skin surfaces with increased collagen synthesis on the panellists that participated as assessed against those who consumed the placebo.

It should be noted that it was unexpectedly discovered that the bioavailability of the active ingredients from 3 different food formats have been tested in this invention (capsules, small drink and a small bar). The relative bioavailability was judged from the steady state plasma concentration at the end of a 3 week dosing study (for vitamin C, lycopene, vitamin E, and DHA/EPA in red blood cells) or from the AUC as obtained after a single dose on day 1 (starts) for the isoflavones. The results of this study showed that there were no differences between the active delivery and results when different product formats were consumed.

1. A composition comprising:
a) a carotenoid;
b) a PPAR ligand;
c) an oestrogen receptor binding agent;
d) an agent involved in post translational modification of collagen; and
e) an antioxidant
wherein the agent involved in post translational modification of collagen and antioxidant are present at a daily dosage amount (x) that is from about 0.02 to about 23 times a daily dosage amount (y) that is the daily dosage amount of carotenoid, PPAR ligand and oestrogen receptor binding agent in the composition further wherein the antioxidant daily dosage amount/carotenoid daily dosage amount within the composition is from about 0.008 to about 300, the composition being one capable of delivering from about 45 to about 2000 mg PPAR ligand, from about 0.5 to about 120 mg carotenoid, from about 8 to about 125 mg oestrogen receptor binding agent, from about 45 to about 1100 mg of an agent involved in post translational modification of collagen and from about 0.85 to about 310 mg antioxidant in about a daily dosage.

2. The composition according to claim 1 wherein the composition is suitable to improve skin characteristics upon consumption.

3. The composition according to claim 1 wherein the composition is from about 40 to about 100% by weight active.

4. The composition according to claim 1 wherein the composition delivers PPAR ligand from about 100 to about 1400 mg, oestrogen receptor binding agent from about 10 to about 70 mg, an agent that is involved post-translational modification of collagen from about 60 to about 400 mg, caretonoid from about 1 to about 60 mg and antioxidant from about 1 to about 175 mg in an about daily dosage.

5. The composition according to claim 1 wherein the composition delivers PPAR ligand from about 400 to about 800 mg, oestrogen receptor binding agent from about 15 to about 45 mg, an agent that is involved post-translational modification of collagen from about 150 to about 250 mg, caretonoid from about 1 to about 10 mg and antioxidant from about 10 to about 45 mg.

6. The composition according to claim 1 wherein the PPAR ligand comprises an omega-3 fatty acid, the oestrogen receptor binding agent comprises an isoflavone, the agent that is involved in the post-translational modification of collagen comprises vitamin C, the carotenoid comprises β-carotene, licopene or both, and the antioxidant comprises vitamin E.

7. The composition according to claim 1 wherein the composition further comprises wax, gum, stabilizer, emulsifier, plant extract, flavouring, preservative, colorant, vitamin A, vitamin A derivative, niacinamide, amino acid or mixtures thereof.

8. The composition according to claim 1 wherein composition is a capsule, tablet, drink or snack bar.

9. The composition according to claim 1 wherein the composition is a food product.

10. A method for improving skin characteristics by consuming the composition of claim 1.

11. The method according to claim 10 wherein the method comprises instructions to consume the composition about daily for about 10 to about 16 weeks.

12. The method according to claim 10 wherein the method comprises instructions to consume the composition daily for about 12 to about 15 weeks.
